(19) United States
(12) Patent Application Publication
(10) Pub. No.: US 2018/0085018 A1
(43) Pub. Date: Mar. 29, 2018

(54) Systems And Methods For Splaying Microelectrode Sensors

(71) Applicant: Paradromics Inc., San Jose, CA (US)

(72) Inventors: Matthew Angle, San Jose, CA (US); Yifan Kong, San Jose, CA (US); Edmund Huber, San Francisco, CA (US)

(73) Assignee: Paradromics Inc., San Jose, CA (US)

(21) Appl. No.: 15/279,429

(22) Filed: Sep. 29, 2016

(51) Int. Cl. A61B 5/0408; A61B 5/0478

(52) U.S. Cl. CPC A61B 5/0408; A61B 2562/125; A61B 5/0478

(57) ABSTRACT

A system and method for creating desired splay patterned microelectrodes is disclosed. A bundle of microwires is arranged into a desired splay pattern. This may be performed mechanically with a rigid frame, electronically by charging the microwires, or with some other technique. The microwires in the desired splay pattern are then heated to release internal tension. Upon completion of heating, the microwires are then slowly cooled such that the splayed microwires will retain the desired splay pattern. Insulation may then be added to the microwires if the microwires are not already insulated.

### TECHNICAL FIELD

The present invention relates to the field of medical electrode sensors. In particular, but not by way of limitation, the present invention discloses techniques for splaying microelectrode sensors.

### BACKGROUND

Modern medicine and medical research use medical electrodes to detect electrical signals within human tissue. The most well-known usage of medical electrodes is as part of an electrocardiogram (ECG or EKG). An electrocardiogram detects and displays the electrical activity of heart tissue and may be used as part of a medical test of a patient's cardiovascular system. The recorded electrical activity may be kept as part of a patient's medical record. The display of the electrical activity appears as a line with spikes and dips that are called waves.

An electrocardiogram senses electrical currents using electrodes placed on the patient's skin. However, due to the not-trivially-reversed distortion effects that electrical signals suffer in the intervening centimeters of tissue and bone between the heart and the skin, for the purposes of accurately and locally sampling electrical currents in heart tissue it may be desirable to insert microelectrodes into the tissue of a patient. In addition, certain symptoms of the brain, spiral cord, muscles, or other soft issues may serve as clinical indications for inserting current-sensing microelectrodes into those tissues for the purpose of ascertaining electrical behavior of those tissues.

To obtain as much electrical activity information as possible, a bundle of microelectrodes may be introduced into the soft tissue of a subject to be tested. However, the doctor or medical researcher will generally wish to minimize the disruption of the subject's soft tissue when the bundle of microelectrodes is inserted into that soft tissue. It would therefore be desirable to implement systems and methods for creating medical microelectrodes that obtain as much electrical activity information as possible while minimizing disruption to the subject's soft tissue.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates a diagrammatic representation of a machine in the example form of a computer system within which a set of instructions, for causing the machine to perform any one or more of the methodologies discussed herein, may be executed.

FIG. 2A illustrates bundle of six microelectrodes within a tube pressed against some soft tissue and ready for insertion.

FIG. 2B illustrates the bundle of six microelectrodes from FIG. 2A inserted into soft tissue with an undesirable close pattern.

FIG. 2C illustrates the bundle of six microelectrodes from FIG. 2A inserted into soft tissue with a desirable splay pattern.

FIG. 3 illustrates a process of creating annealed splayed microwires for use as microelectrodes.

FIG. 4 illustrates a splayed bundle of microwires being heated in an oven to perform annealing.

FIG. 5 illustrates an example of rigid frame guide that is used to guide individual microwires in a microwire bundle into a desired pattern.

FIG. 6A illustrates a bundle of microwires in a tube with a length of microwire extending out of the tube.

FIG. 6B illustrates the bundle of microwires from FIG. 6A after a high voltage potential has been applied to the microwire bundle thus forcing the microwires to repel each other.

FIG. 7 illustrates a shaped counter electrode used to attract the ends of the microwires in a bundle.

FIG. 8 illustrates a process of creating annealed splayed microwires for use as microelectrodes that performs the annealing process before insulation is placed on the microwires.

The Figures depict various embodiments for purposes of illustration only. One skilled in the art will readily recognize from the following discussion that other embodiments of the structures and methods illustrated herein may be employed without departing from the described principles.

### DETAILED DESCRIPTION

The following detailed description includes references to the accompanying drawings, which form a part of the detailed description. The drawings show illustrations in accordance with example embodiments. These embodiments, which are also referred to herein as “examples,” are described in enough detail to enable those skilled in the art to practice the invention. It will be apparent to one skilled in the art that specific details in the example embodiments are not required in order to practice the present invention. For example, although some example embodiments are disclosed with reference to creating microelectrodes for brain tissue, the same techniques can be used to test other types of soft tissue. The example embodiments may be combined, other embodiments may be utilized, or structural, logical and electrical changes may be made without departing from the scope what is claimed. The following detailed description is, therefore, not to be taken in a limiting sense, and the scope is defined by the appended claims and their equivalents.

In this document, the terms “a” or “an” are used, as is common in patent documents, to include one or more than one. In this document, the term “or” is used to refer to a nonexclusive or, such that “A or B” includes “A but not B,” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

Computer Systems

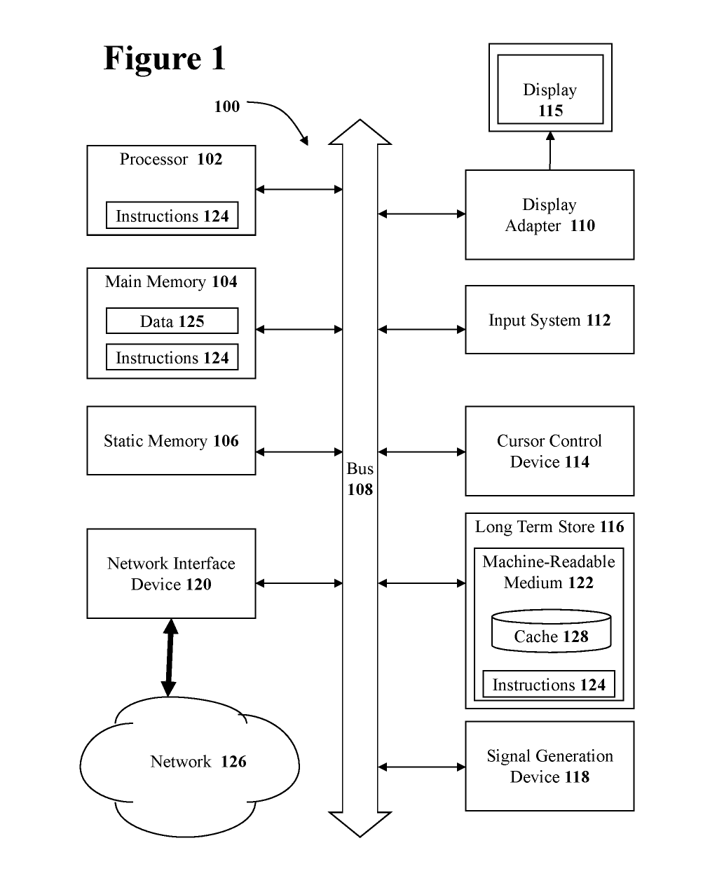

FIG. 1 illustrates a diagrammatic representation of a machine in the example form of a computer system 100 that may be used to implement portions of the present disclosure. Within computer system 100 there are a set of instructions 124 that may be executed for causing the machine to perform any one or more of the methodologies discussed herein. In a networked deployment, the machine may operate in the capacity of a server machine or a client machine in client-server network environment, or as a peer machine in a peer-to-peer (or distributed) network environment. The machine may be a small card, personal computer (PC), a tablet PC, a set-top box (STB), a Personal Digital Assistant (PDA), a cellular telephone, a web appliance, a network router, switch or bridge, or any machine capable of executing a set of computer instructions (sequential or otherwise) that specify actions to be taken by that machine. Furthermore, while only a single machine is illustrated, the term “machine” shall also be taken to include any collection of machines that individually or jointly execute a set (or multiple sets) of instructions to perform any one or more of the methodologies discussed herein.

The example computer system 100 includes a processor 102 (e.g., a central processing unit (CPU), a graphics processing unit (GPU) or both), a main memory 104 and a static memory 106, which communicate with each other via a bus 108. The computer system 100 may further include a display adapter 110 that drives a display system 115 such as a Liquid Crystal Display (LCD), Cathode Ray Tube (CRT), or other suitable display system. The computer system 100 includes an input system 112. The input system may handle typical user input devices such as a keyboard. However the input system may also be any type of data acquisition system such an analog-to-digital (A/D) converter. The computer system 100 may also include, a cursor control device 114 (e.g., a trackpad, mouse, or trackball), a long term storage unit 116, an output signal generation device 118, and a network interface device 120.

The long term storage unit 116 includes a machine-readable medium 122 on which is stored one or more sets of computer instructions and data structures (e.g., instructions 124 also known as ‘software’) embodying or utilized by any one or more of the methodologies or functions described herein. The instructions 124 may also reside, completely or at least partially, within the main memory 104 and/or within the processor 102 during execution thereof by the computer system 100, the main memory 104 and the processor 102 also constituting machine-readable media. Note that the example computer system 100 illustrates only one possible example and that other computers may not have all of the components illustrated in FIG. 1 or may have additional components as needed.

The instructions 124 may further be transmitted or received over a computer network 126 via the network interface device 120. Such transmissions may occur utilizing any one of a number of well-known transfer protocols such as the File Transport Protocol (FTP). The network interface device 120 may comprise one or more wireless network interfaces such as Wi-Fi, cellular telephone network interfaces, Bluetooth, Bluetooth LE, Near Field Communication (NFC), etc.

While the machine-readable medium 122 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies described herein, or that is capable of storing, encoding or carrying data structures utilized by or associated with such a set of instructions. The term “machine-readable medium” shall accordingly be taken to include, but not be limited to, solid-state memories, flash memory, optical media, and magnetic media.

For the purposes of this specification, the term “module” includes an identifiable portion of code, computational or executable instructions, data, or computational object to achieve a particular function, operation, processing, or procedure. A module need not be implemented in software; a module may be implemented in software, hardware/circuitry, or a combination of software and hardware.

In the present disclosure, a computer system may comprise a very small microcontroller system. A microcontroller may comprise a single integrated circuit that contains the four main components that create a computer system: an arithmetic and logic unit (ALU), a control unit, a memory system, and an input and output system (collectively termed I/O). Microcontrollers are very small and inexpensive integrated circuits that are very often used within digital electronic devices. A microcontroller may be integrated along with other functions to create a system on a chip (SOC).

Medical Microelectrodes Overview

In certain situations it can be desirable to physically insert electrodes within soft tissue in order to detect electrical activity within that soft tissue. However, the insertion of microelectrodes is an invasive procedure that affects the soft tissue. To minimize the disruption of the soft tissue, very small microwire-based microelectrodes may be used. A microwire based microelectrode consists of an insulated microwire with an exposed conductor at the end of the microwire that acts as the electrode.

To maximize the amount of electrical activity information collected within soft tissue, a bundle of many microwire-based microelectrodes may be inserted into the soft tissue. In this manner, the electrical activity at the end of each individual microelectrode may be detected and recorded. However, one problem faced by the insertion of bundles of microwire based microelectrodes into soft tissue is obtaining a desirable final location of the individual microelectrodes post insertion.

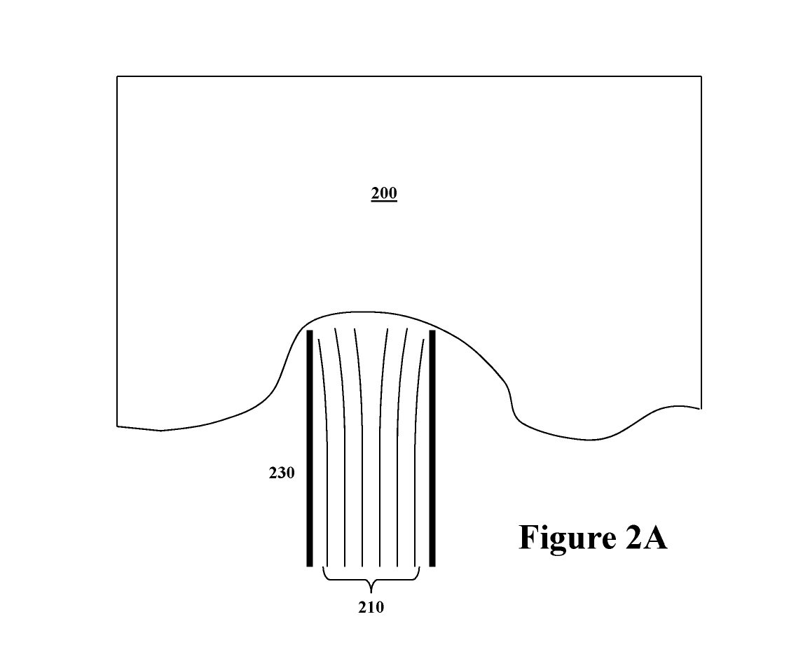

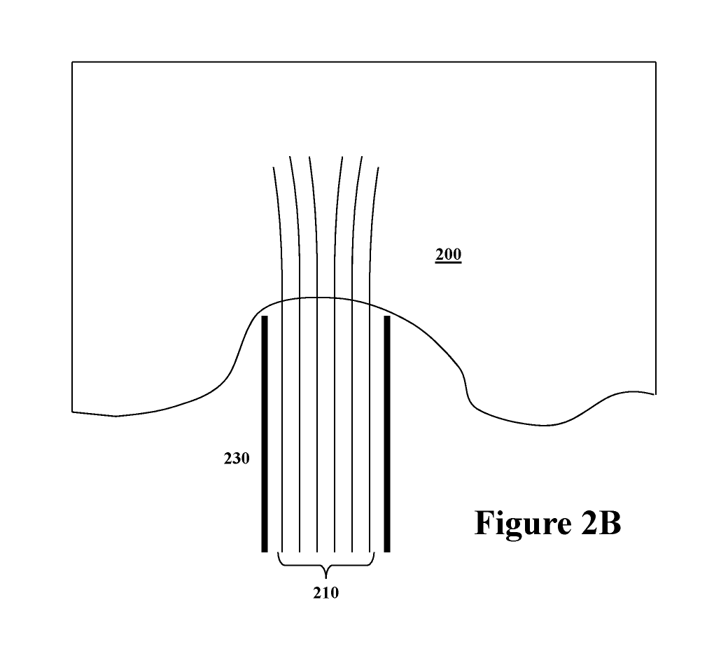

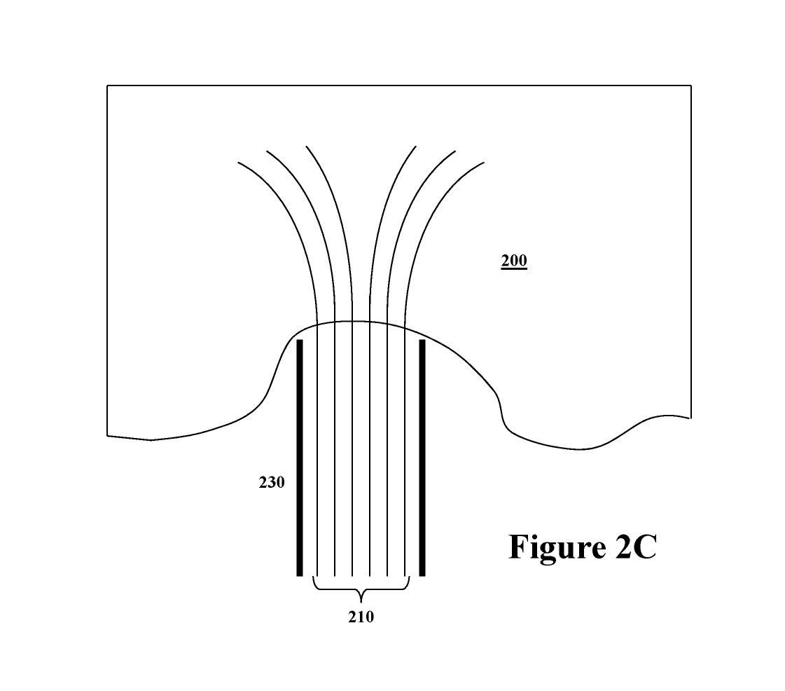

FIG. 2A illustrates an example bundle of six microwire based microelectrodes 210 within a tube 230. The tube 230 containing the bundle of microelectrodes 210 ready for insertion is pressed against some soft tissue 200. For clarity of the diagrams, the diagrams of 2A to 2C illustrate only six microelectrodes but most implementations would generally have many more microelectrodes in a bundle in order to obtain more electrical activity information.

The insertion of the microelectrodes takes place by pushing the bundle of microwire based microelectrodes 210 within tube 230 into the soft tissue 200 such that the microwires become unbound as the microelectrodes 210 enter the soft tissue 200. A simple bundle of microwire based microelectrodes inserted into soft tissue 200 will tend to spread only minimally as illustrated in FIG. 2B.

The small spread of microelectrodes as illustrated in FIG. 2B is a problem for at least two reasons. First, a cluster of microwires occupying a small volume of tissue can cause greater disruption to the soft tissue 200 than if the microwires were spread out into a larger volume of the soft tissue 200. Secondly, it would be very desirable if the ends of microwire microelectrodes 210 were at a regular or controllable distance apart. The second property is especially important for microelectrode insertion into brain tissue since a regular dispersion of electrically conductive microelectrodes into the brain tissue allows for regular recording of a variety of locations and thus avoiding issues of oversampling from microelectrode “clumping” and missed samples from voids in the original microwire bundle.

To reduce disruption to the soft tissue 200 and to obtain a better collection of electrical activity information, a wider spread of microelectrodes is much more desirable. For example, FIG. 2C illustrates a wider splay of microelectrodes into the soft tissue 200. To obtain the desired splay of FIG. 2C, the microwires 210 should have potential energy in spring form such that when the microwires 210 are pushed out of the tube 230, the microwires 210 release this spring energy by spreading out to a relaxed state during the insertion thereby forming the splay pattern of FIG. 2C.

Medical Microelectrode Manufacturing

The various processes and techniques for obtaining the desired microwire splaying (such as displayed in FIG. 2C) are very limited because this microwire-based microelectrode technology is not widely used for tissue implants. Thus, the present disclosure proposes methods for microwire modification whereby a straight bundle of microwires is first pre-arranged into a desired splay pattern and then thermally annealed to make the splay pattern of the microwire bundle permanent. The thermal annealing relaxes the tension within the microwires such that the microwire splay pattern becomes the natural low energy state of the individual microwires in the microwire bundle.

After the annealing process, the annealed microwires may be drawn into a tube or needle which causes the individual microwires to obtain potential energy in spring form. When the tube or needle containing the microwire bundle is then placed against a soft tissue surface (as illustrated in FIG. 2A) and the annealed microwire bundle is pushed out from the tube or needle 230 and into the soft tissue 200, the annealed microwires 210 will attempt to return to their relaxed state as they exit the tube thereby creating the desired splay pattern as illustrated in FIG. 2C.

The specific annealing process of these microwires will primarily depend on what specific insulator and conductor materials the microwires are made of. The maximum splaying of the microwires is determined by their ability to elastically deform, which is their ability to be displaced and when released, return to their lowest stress state. The materials used to construct the microwire are responsible for this property. For example, microwires constructed with insulation such as glass may have a lower ability to elastically deform than microwires constructed with an insulated polymer coating. Thus, the annealing process for glass-coated microwires does not allow for as acute splaying angles as the annealing process for polymer-coated microwires.

As the insulation of a microwire is typically the volumetrically larger material than the conductor core of the microwire and the insulation has a larger moment of inertia compared to the conductor core, the microwire insulation properties will often have a greater effect on the microwire's mechanical properties. Of course, it is possible to produce microwires with a larger conductor core and thin insulation, in which case this may no longer hold true.

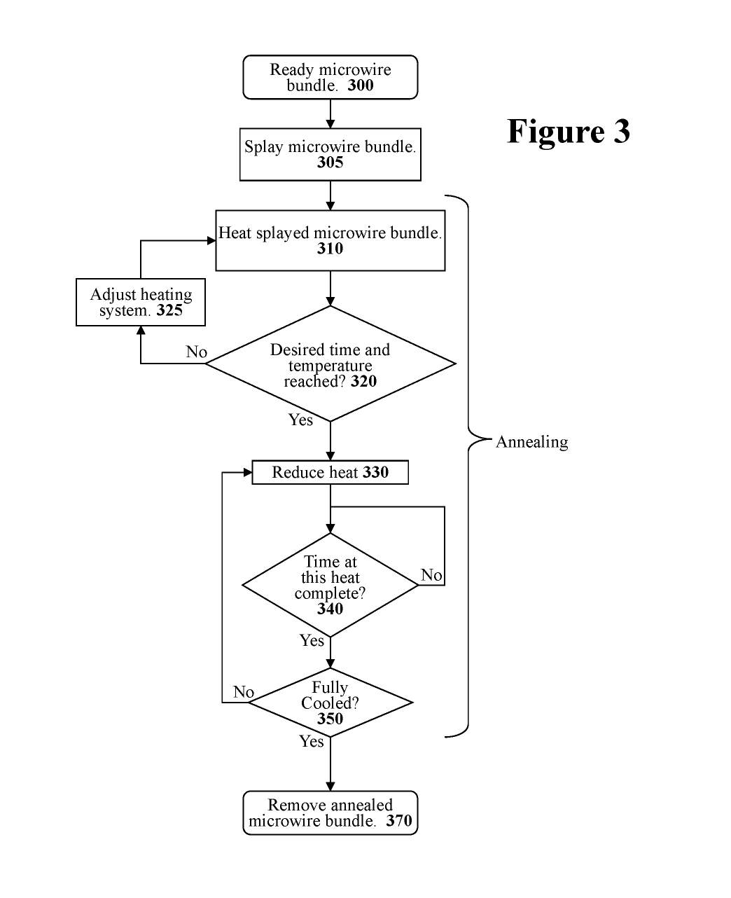

FIG. 3 illustrates the process of creating annealed splayed microwires for use as microelectrodes. First a microwire bundle of parallel microwires is created at stage 300. Next, at stage 305, the microwire bundle is arranged into a desired splayed pattern. Several different techniques may be used to create the desired splay pattern of the microwire bundle. In one simple technique, the microwires are mechanically splayed using a pattern mold that physically holds the microwires from the bundle in specific positions. Several additional techniques for splaying microwire bundles will be discussed in later sections of this disclosure document.

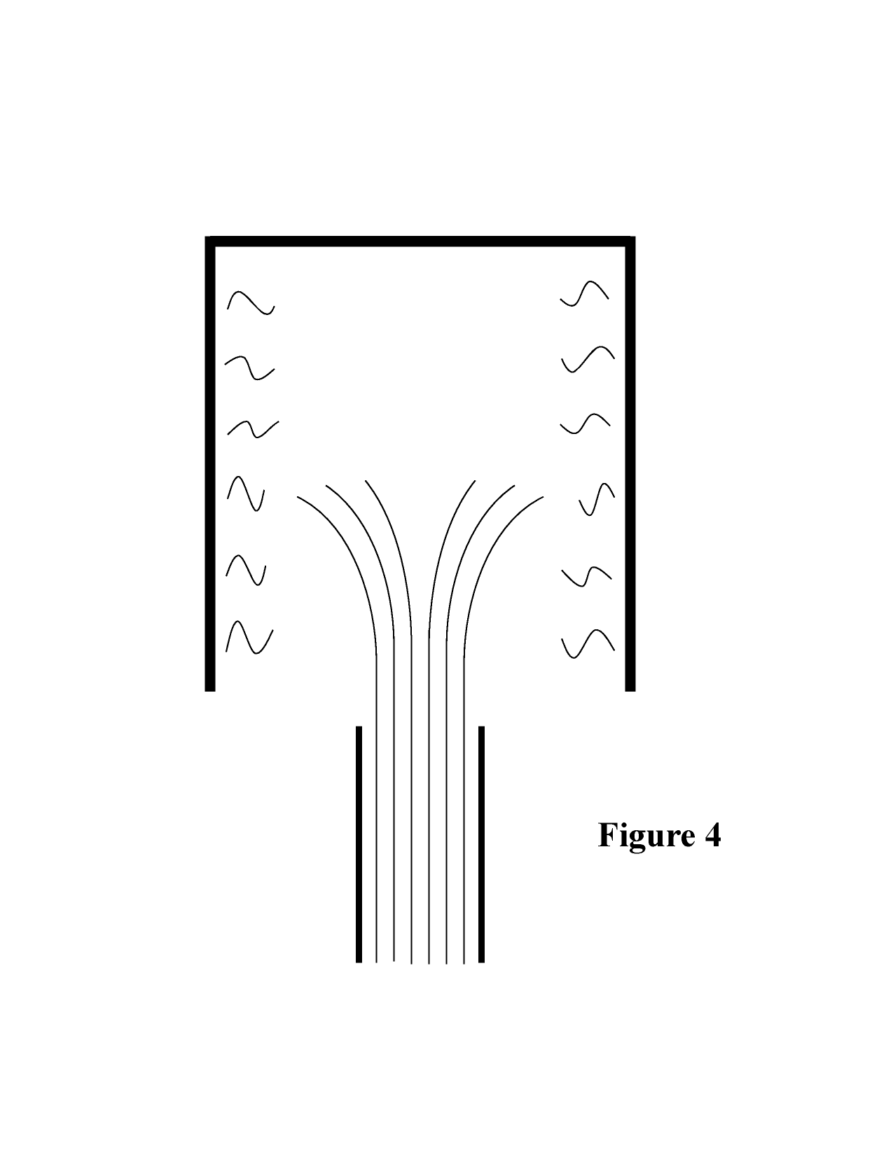

After putting the microwires into a splayed configuration pattern, the annealing process of the splayed microwires begins. Thus, at stage 310 the splayed microwire bundle is heated. The heating process may be performed with a resistive heating element, by induction heating, or with any other suitable heating system. FIG. 4 illustrates a splayed microwire bundle being heated in an oven.

To obtain very consistent quality, a computer system 100 may be used to control the heating system that is being used to heat the microwire bundle. The computer system may carefully control the temperature and the time of the heating process with a feedback control system. Thus, at stage 320 the control system checks the temperature and elapsed time of the microwire heating process. If the heating is not complete then the control system will adjust the heating system as necessary at stage 325 and continue heating the splayed microwire bundle, returning to 310.

The annealing of microwires can be done in a variety of equipment systems that are capable of reaching the high temperatures required (up to 1000 degrees Celsius). One possibility is to use a furnace or an oven to anneal the microwire bundle. Another possibility is to use a resistive loop heater and slowly draw the heater along each microwire. This technique will also produce thermal gradients similar to floating zone refining. Another possibility is to use inductive heating to heat the microwires similar to the technique used in the Taylor-Ulitovsky process for microwire pulling. Laser heating of microwires is also a possible heating solution. Laser heating can create precisely controlled thermal gradients in different regions along the microwire that could force independent stresses and also act as the method of splaying during annealing simultaneously. Heating via other radiation methods that are angle dependent (such as by polarized waves) could also allow for preferential heating of wire splayed in certain directions and not others.

As previously mentioned, annealing temperature requires precise control in order to avoid introducing defects and fragility into the microwire. For typical annealing processes, a glass material must be heated to its annealing point and held such that any mechanical stress within the glass can be released. The annealing point of glass materials varies based on the specific glass composition but typically ranges from 400 degrees Celsius to 1200 degrees Celsius and can be lower or higher depending on the annealing speed required. For microwires, a lower annealing point might be used in some cases due to the thinness of the glass insulation layer necessitating lower amounts of time for stress relaxation. The time the microwire must be held at the annealing temperature varies but may range from 10 minutes or less at high temperatures, to days at very low temperatures. These types of extremes may be necessary as the microwires are of multi-part composition. For example it might be necessary for the glass layer to be heated at a lower temperature so not to melt a low-melting-point metal that forms the core and therefore a longer time is required to anneal the glass layer.

Returning back to FIG. 3, after the desired temperature and heating time have been completed at stage 320 the annealing system proceeds to stage 330 to begin cooling the microwires. The cooling rate of the annealing stage depends on the material thickness. For a single microwire, the cooling rate can be very high with a rate of 100 C/min or more due to the very small size of the microwire allowing for fast heat transfer to the environment. However, for microwires which are bundled together and splayed the heating rate must be calculated from the total diameter of the bundle. With a 1 cm diameter bundle the cooling might be done on the order of 5 degrees Celsius per minute.

Any additional supports for the splaying bundle while heating must be included in this heating rate so it could be significantly lower. The cooling range should span from the annealing temperature to the strain point of the glass, typically a window of 100 degrees Celsius or 200 degrees Celsius. These cooling rates can vary particularly in the case of a large inner metallic core wire and a thin insulating layer, as the large inner metallic core will transport heat more effectively and distribute thermal gradients. The parameters for the cooling of a microwire bundle will differ from the parameters used for pure glass.

As with the heating process, the cooling process may be controlled by computer system 100 in order to carefully cool the microwires. At step 330, the temperature is reduced to a first cooling level. Stage 340 then keeps the temperature at that cooling level for a specified amount of time. Next at stage 350 the control system determines if the cooling process is complete. If the cooling is not yet complete, the system returns to stage 330 to reduce cooling temperature and then hold it at that reduced cooling temperature for a specified amount of time at stage 340. This process repeats through a specified amount of iterations until the microwires are fully cool at stage 350. The microwire bundle can then be removed from the annealing system at stage 370.

The annealing process may be conducted in an inert gas or in vacuum. If the annealing is conducted in an inert gas such as argon, the annealing process has the advantage of avoiding the formation of additional metal oxides at the microwire tips at high temperatures. If the microwire insulation is a polymer or an organic material then using an inert gas or vacuum avoids high temperature oxygen based decomposition of the insulation that occurs at a lower temperature than pure thermal decomposition thereby extending the usable temperature range of this method. If annealing is performed in a vacuum, the annealing process must be carried out much more slowly due to the reduced thermal coupling between the heater elements and the microwires. One possibility for avoiding this is to apply heat on the microwire bundle supports such that the heating is done by conduction through the wire core in the absence of convection. This heating method must also be done more slowly than by convection since the heat will have to propagate up the microwires.

Polymer materials may or may not be able to be thermally annealed depending on their composition. Generally, for microwires produced by a thermal drawing process it should be possible to reheat those polymers to anneal them into a new low-stress arrangement.

Mechanical Splaying of Microwires

Referring to back to stage 305 of FIG. 3, before a microwire bundle is annealed, the microwire bundle must be put into the desired splay pattern. The pre-splaying of microwires can be accomplished by several different methods. The fundamental issue is to splay the microwires in a controlled manner and the splay method must be capable of sustaining the extreme heat necessary for annealing the microwires in the microwire bundle.

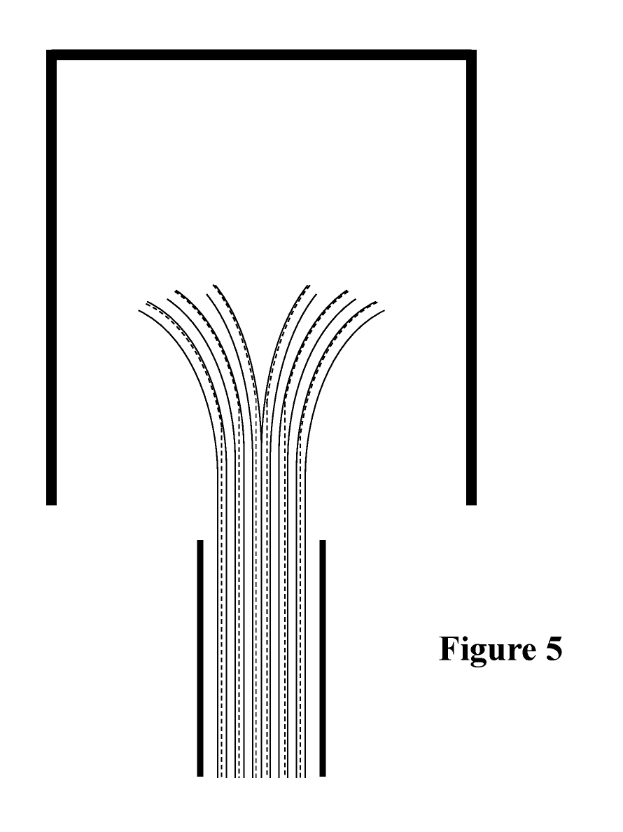

A simple method of splaying the microwire bundle is to physically hold the splayed microwires in a desired position in a mechanical manner. This method necessitates a physical guidance of each wire. This may be accomplished by inserting the microwires into a physical rigid frame guide. FIG. 5 illustrates an example of rigid frame guide that is used to guide individual microwires of a microwire bundle into a desired splay pattern. The rigid frame guide holds the microwires physically in position for annealing.

Electrical Charge to Splay Microwires

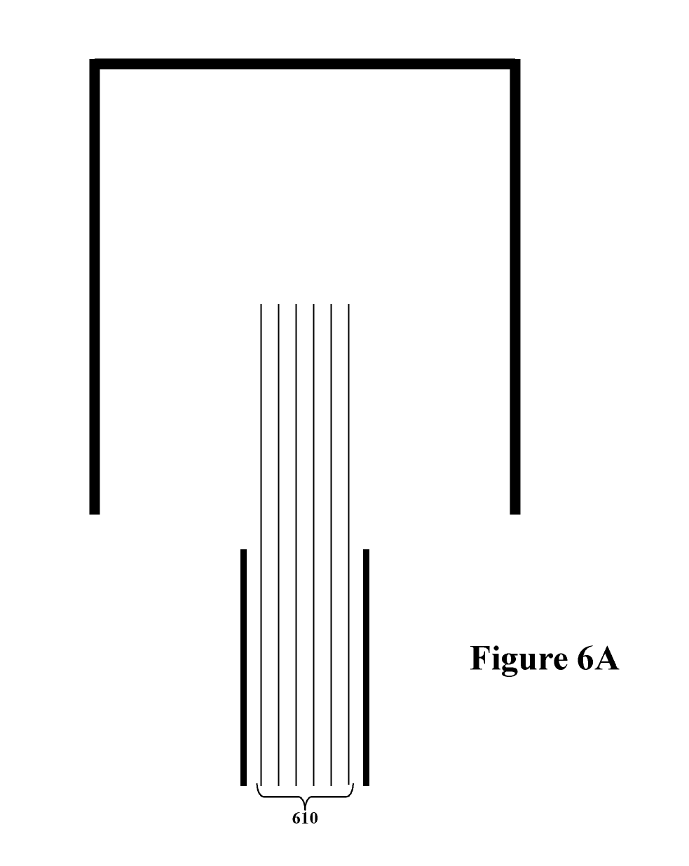

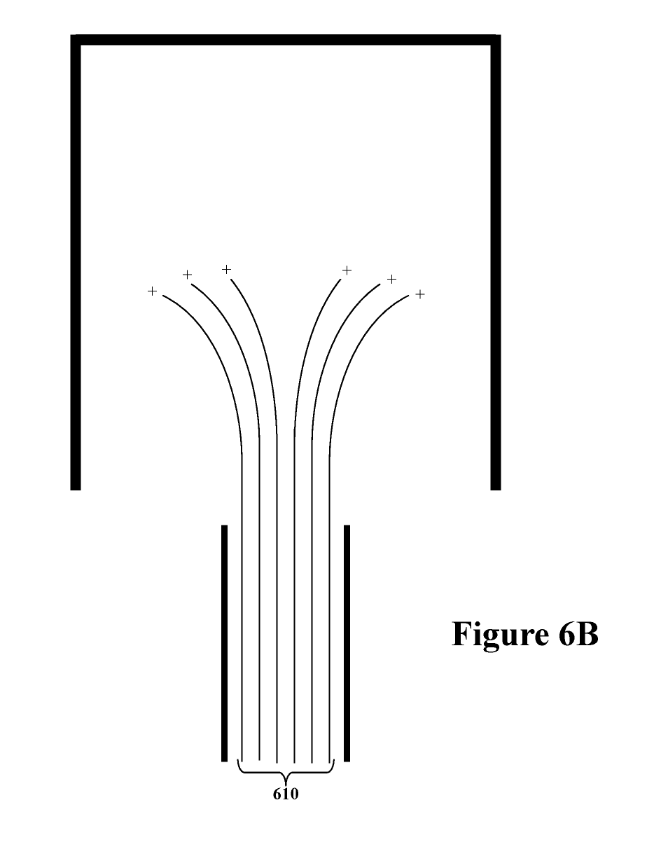

Another method of splaying microwires is to use electrical charge to force the individual microwires away from each other thus creating a splay pattern of microwires repelled from each other. To perform this technique, first a bundle of microwires is placed into a tube with a length of microwires extending out of the tube. An example of this is illustrated in FIG. 6A. The other ends of the microwires in the bundle 610 are electrically connected together. One method performing this electrical connection is by physical vapor deposition of a metal on that end. Another method of electrically connecting the microwires is to use electroplating to connect all the microwires together.

Once this is done, a high voltage is applied to all the microwires in the microwire bundle at end 610. The high voltage charges the microwires and the free charge at the end of the microwires forces the free ends of the microwires apart. This is illustrated in Figure 6B wherein the charge on the individual microwires forces the microwires apart from each other due to positive charges repelling each other. The high voltage and charge are then held while the microwire bundle is annealed. This method can be used to change the splay shape in a controllable manner by varying the voltage applied to all the microwires, thereby controlling the distance between each microwire in the bundle.

Varied Voltage Electrical Charge to Splay Microwires

In the technique described in the previous section all of the microwires are charged up with a common high voltage to spread the microwires. Another possibility is to alter the voltage on each individual microwire of the microwire bundle. Individually altering the voltage on each microwire will allow many different splay patterns to be created. However, this method has limitations since the voltage cannot be altered so much that it causes dielectric breakdown between microwires.

Shaped Electrical Charge to Splay Microwires

The previous two sections described how electrical charge can be used to create a splay patter in a bundle of microwires. However, these techniques can be further refined to create controlled desired splay patterns. Specifically, the splaying pattern may be controlled by putting a shaped ground or negatively charged counter electrode proximate to the free ends of the microwires. In this manner, the positively charged microwires will be attracted to features on the grounded or negatively charged shaped counter electrode. Thus, the microwires will organize to reflect the changes in electric field between the microwire tips and features on the shaped counter electrode.

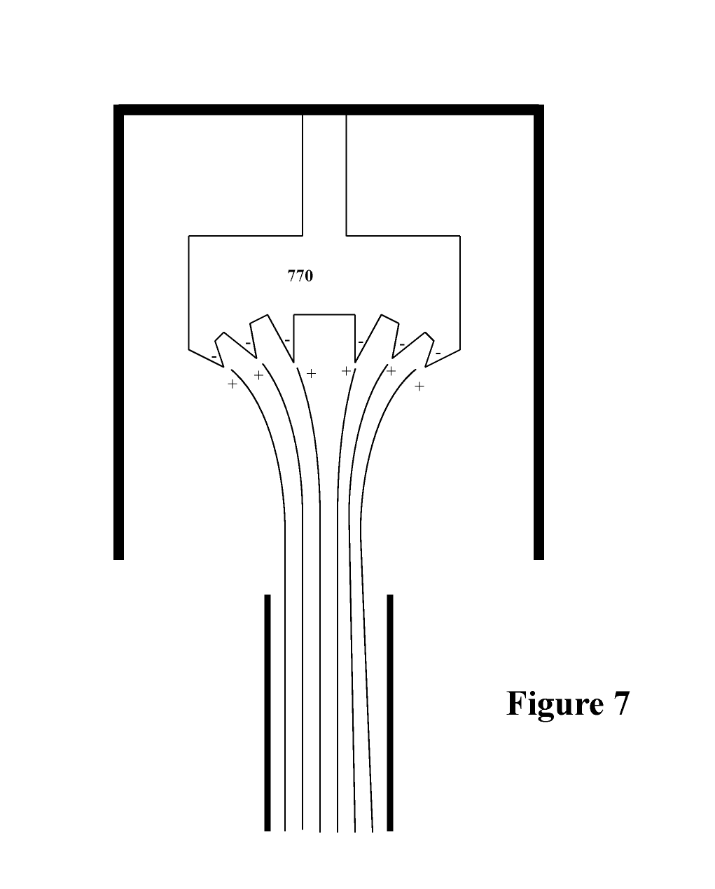

FIG. 7 illustrates an example of a shaped counter electrode used to shape microwires into a desired splay pattern. The shaped counter electrode 770 is placed within a heating system for annealing a bundle of microwires. As illustrated in FIG. 7, the ends of the microwires are attracted to the tips on the shaped counter electrode 770 when the microwires are positively charged thus causing the microwire bundle to form a splay pattern that is controlled by the specific physical features of the shaped counter electrode 770.

The method of splaying microwires by applying a shaped counter electrode must account for both the changed mechanical properties of the microwire at high annealing temperatures (i.e. the softening of the strains from bending) as well as the self-interaction from many microwires. This shaped counter electrode technique may be used in conjunction with the application of a different voltage on different microwires or sets of microwires, so as to double the usable range of voltages without reaching dielectric breakdown or arcing of current between electrodes.

The voltages used for these electric charge based spreading methods might range between 100 Volts to 30000 Kilovolts. However, the application of a very high voltage is less desirable in the case of shaped counter electrodes or microwires held at different potentials for the aforementioned reasons of dielectric breakdown.

Another issue is that at high voltages, a shaped counter electrode will have a more uniform field and any voltage differences are generally as a percentage of the held voltage. While both the microwires and the shaped counter electrode can be held at either potential, it is more preferable to hold the wire electrode at a positive voltage and the counter electrode at a negative voltage. This is to avoid field emission of the material if the entire process is held under vacuum.

The various high-voltage based splaying systems may operate well in a vacuum. The advantage of doing a high-voltage based splaying method within a vacuum is that a dielectric gas can be avoided that might cause breakdown otherwise. Field emission can still occur, but is mitigated by holding the microwire at positive potentials and making the counter electrode smooth, such that a more uniform electric field builds up on the shaped counter electrode. In vacuum, the upper range of applicable voltage may be much higher and can be greater than 30000 Kilovolts.

The splaying of the microwires need not force the microwires to all go in different directions. The splaying pattern could all have a generalized curvature in one direction or a few directions. This might be used to allow microwires to curve and navigate complex geometries such as around the vasculature of tissue or into deep regions of tissue while avoiding some others. The method of splaying wires in this case may be done mechanically by putting the microwires in a curved tube during annealing. The shaped counter electrode for high voltage microwire splaying could also be used to create complex curvature as well as splaying at one end.

An alternative method is to add charge onto the insulating coating of the microwires by means of ionized gas or exposure of the insulator to high voltages or a triboelectric effect. This method would not necessitate the electrical connection of all the conductive microwire cores but the splaying control is more limited in this method as the charge deposition on the insulator jacket would be more difficult to control precisely.

Insulating Microwires After Splaying

Some microwires may not have insulation that can handle the intense heat that may be required during the annealing process. For example, some polymer insulation materials will thermally degrade before the microwires can be properly annealed. Thus, to create splayed microwires with those types of temperature sensitive polymer insulation materials, the polymer insulation material must be applied to the metal microwire core after the microwire has been through the annealing process.

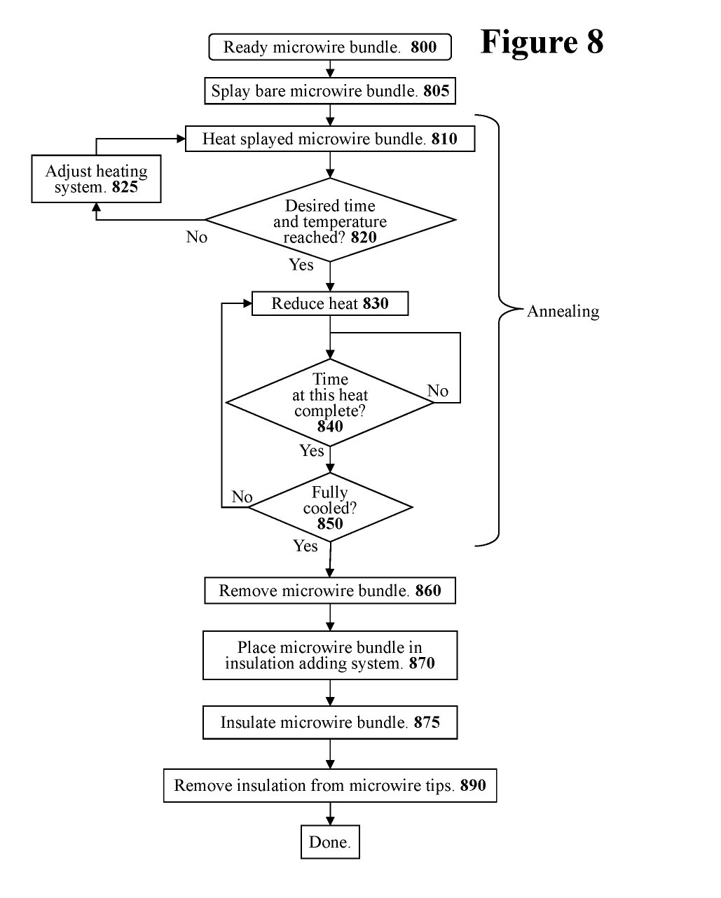

FIG. 8 illustrates a process for creating splayed microelectrodes with polymer insulation materials that cannot withstand high heat. After readying a bare (no insulation) microwire bundle at stage 805, that bare microwire bundle is then splayed into the desired splay pattern at stage 805. This may be performed with any of the splaying techniques disclosed in the previous three sections of this document.

Next, starting at stage 810 and continuing through to stage 850, the process may then use the same annealing process disclosed in stages 310 to 350 in the method illustrated in FIG. 3. The annealing process puts the splayed bare microwires into an unstressed state such that the splay pattern of the bare microwires will become the default shape of the bare microwires.

After the annealing, the bare microwire bundle is removed from the annealing system at stage 860. Next, at stage 870, the splayed microwire bundle is places into an insulation adding system. The splayed microwire bundle is then insulated at stage 875. This might be performed, for example, by one of many different techniques used to deposit an insulator material on the annealed bare microwires. This technique allows for the use of thermoset polymers that cannot be thermally annealed.

Another possibility is to deposit materials created by gas or liquid phase vapor deposition post wire drawing. For polymers that are deposited by thermoset or by ceramics or metals deposited by vapor, one possibility is to use a liquid based polymerization method. This technique would allow for microwires to be pre-splayed and then a polymer or other insulator layer to be deposited by electroless plating by dipping the splayed microwires into solution or splaying them while in this solution. The microwires could then be removed when a sufficiently thick layer was deposited, or the deposition could be self-limiting by the use of a multistage process including a sensitization layer as typically used in electroless deposition. Deposition using electric current (i.e. Electroplating) could also be accomplished for certain materials by using an electric current on a pre-splayed bundle to catalyze a surface reaction. Such a reaction would be surface limited due to the insulating nature of the deposited material.

A versatile alternative deposition method for a pre-splayed bundle is gas-phase deposition, such as with the gas phase deposition of paralyene on splayed microwires, as well as chemical vapor deposition and atomic layer deposition coatings of ceramics such as alumina or hafnium, or even for plasma assisted deposition of layers including metals. For gas phase deposition strategies it is generally necessary that the bundle be splayed in a vacuum chamber or chamber of inert gas.

Other deposition techniques such as sputtering or physical vapor deposition of insulators are not as conformal, but in principle might be used for splayed microwire coating as well. One aspect of gas phase coating, especially for physical vapor deposition and chemical vapor deposition, is the intrinsic stress of the deposited materials which must be accounted for in the deposition parameters. In principle such deposition and stress should be conformal against all sides of a microwire. However non-uniformities in the deposition process would need to be tightly controlled in order to assure that the pre-splayed structure of the microwire bundle would be the final microwire bundle shape. Alternatively, it is conceivable that an unsplayed but free microwire bundle could have intrinsically stressed materials deposited as a nonuniform coating, and the intrinsic stress of the coating itself causes the bundle to preferentially splay.

After insulating the microwires at stage 875, the microwires may be removed from the insulation adding system. Next, a final stage 890 is to remove the insulation material from the microwire tips at the end of the splay pattern. This creates exposed bare microwire conductor to serve as microelectrodes.

The preceding technical disclosure is intended to be illustrative, and not restrictive. For example, the above-described embodiments (or one or more aspects thereof) may be used in combination with each other. Other embodiments will be apparent to those of skill in the art upon reviewing the above description. The scope of the claims should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to comply with 37 C.F.R. §1.72(b), which requires that it allow the reader to quickly ascertain the nature of the technical disclosure. The abstract is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment.

What is claimed is:

1. A manufacturing system for manufacturing microelectrodes, said manufacturing system comprising the elements of:
a splaying system that splays the ends of a plurality of microwires in a microwire bundle into a desired splay pattern;
a heating system, said heating system to heat said plurality of microwires in said microwire bundle to release internal tension in said plurality of microwires; and
a cooling system to slowly cool said plurality of microwires in said microwire bundle to retain said desired splay pattern.

2. The manufacturing system for manufacturing microelectrodes as set forth in claim 1 wherein said splaying system comprises a rigid frame to hold said plurality of microwires in said desired splay pattern.

3. The manufacturing system for manufacturing microelectrodes as set forth in claim 1 wherein said splaying system comprises a tube to hold said plurality of microwires and a high-voltage source to charge said plurality of microwires thereby creating said desired splay pattern.

4. The manufacturing system for manufacturing microelectrodes as set forth in claim 3 wherein said splaying system further comprises a shaped counter electrode to attract said microwires.

5. The manufacturing system for manufacturing microelectrodes as set forth in claim 4 wherein said shaped counter electrode is negatively charged to better attract said microwires.

6. The manufacturing system for manufacturing microelectrodes as set forth in claim 1 wherein said heating system comprises an oven.

7. The manufacturing system for manufacturing microelectrodes as set forth in claim 1 wherein said heating system comprises a resistive loop heater.

8. The manufacturing system for manufacturing microelectrodes as set forth in claim 1 wherein said heating system comprises an induction heating system.

8. The manufacturing system for manufacturing microelectrodes as set forth in claim 1 wherein said heating system comprises a laser heating system.

9. The manufacturing system for manufacturing microelectrodes as set forth in claim 1, said manufacturing system further comprising the elements of:
a computer control system, said computer control system for controlling said heating system and said cooling system.

10. The manufacturing system for manufacturing microelectrodes as set forth in claim 1, said manufacturing system further comprising the elements of:
an insulating system, said insulating system for adding insulation to said plurality of microwires in said microwire bundle.

11. A method for manufacturing microelectrodes, said manufacturing method comprising the stages of:
splaying the ends of a plurality of microwires in a microwire bundle into a desired splay pattern;
heating said plurality of microwires in said microwire bundle to release internal tension within said plurality of microwires; and
cooling said plurality of microwires in said microwire bundle to retain said desired splayed pattern.

12. The method for manufacturing microelectrodes as set forth in claim 11 wherein said splaying is performed with a rigid frame to hold said plurality of microwires in said desired splay pattern.

13. The method for manufacturing microelectrodes as set forth in claim 11 wherein said splaying comprises:
holding said to hold said plurality of microwires in a tube; and
charging said plurality of microwires with a high-voltage source thereby creating a splay pattern.

14. The method for manufacturing microelectrodes as set forth in claim 11 wherein said splaying further comprises:
placing a shaped counter electrode proximate to said plurality of microwires to attract said plurality of microwires.

15. The method for manufacturing microelectrodes as set forth in claim 14 wherein said splaying further comprises:
negatively charging said shaped counter electrode proximate to said plurality of microwires to attract said plurality of microwires.

16. The method for manufacturing microelectrodes as set forth in claim 11, said method further comprising:
controlling said heating and said cooling with a computer control system.

17. A method for manufacturing microelectrodes, said manufacturing method comprising the stages of:
splaying the ends of a plurality of bare microwires in a microwire bundle into a desired splay pattern;
heating said plurality of bare microwires in said microwire bundle to release internal tension within said plurality of microwires;
cooling said plurality of bare microwires in said microwire bundle to retain said desired splayed pattern; and
insulating said bare microwires in said desired splay pattern

18. The method for manufacturing microelectrodes as set forth in claim 17 wherein said splaying is performed with a rigid frame to hold said plurality of microwires in said desired splay pattern.

19. The method for manufacturing microelectrodes as set forth in claim 17 wherein said splaying comprises:
holding said to hold said plurality of microwires in a tube; and
charging said plurality of microwires with a high-voltage source thereby creating a splay pattern.

20. The method for manufacturing microelectrodes as set forth in claim 17 wherein insulating said bare microwires comprises chemical vapor deposition onto said bare microwires.
